(19) United States
(12) Patent Application Publication
(10) Pub. No.: US 2018/0363422 A1
(43) Pub. Date: Dec. 20, 2018

(54) FLUID CHEMISTRY APPARATUS, SYSTEMS, AND RELATED METHODS

(71) Applicant: RESTREAM SOLUTIONS, LLC, Galveston, TX (US)

(72) Inventors: William Wythe Roberts, IV, Galveston, TX (US); Alex Edward Winter, Wimberley, TX (US)

(21) Appl. No.: 15/998,790

(22) Filed: Aug. 16, 2018

Related U.S. Application Data
(63) Continuation-in-part of application No. 15/294,407, filed on Oct. 14, 2016, now abandoned.
(60) Provisional application No. 62/296,901, filed on Feb. 18, 2016.
(60) Provisional application No. 62/546,660, filed on Aug. 17, 2017.

(51) Int. Cl. E21B 41/00; G01N 33/18; G01N 33/28; E21B 47/00; E21B 47/06

(52) U.S. Cl. CPC E21B 41/00; G01N 33/1893; G01N 33/2823; E21B 43/267; E21B 47/06; E21B 47/065; E21B 47/00

(57) ABSTRACT

Systems, apparatus, and methods for duplicating a shear rate of a fluid in a process system and for sensing one or more properties at that shear rate. In some embodiments the process system is a blending system for frack water. In some embodiments, the systems comprise two spools: one spool containing a pH sensor and an oxidation reduction potential sensor whereby a controller senses the corresponding properties of the water therein. And whereby, if her sensed property is outside of a corresponding user selected range, the controller closes a valve to isolate the system from the process. Note that that systems of embodiments further comprise flow control valves to set a flowrate in the system to a value consistent with match a share rate in a process system with which the system might be in fluid communication.

### CROSS REFERENCE TO RELATED APPLICATIONS

This application is a continuation-in-part of application Ser. No. 15/294,407, filed Oct. 14, 2016 and also claims priority to provisional Application Ser. No. 62/546,660, filed on Aug. 17, 2017, titled Hydro-Fracturing Fluid Modeling and Chemistry Application Control Device.

### BACKGROUND

American energy independence, a goal that is tantalizingly close as of this writing, depends to a large extent on the ability of the United States to produce oil (and/or other hydrocarbons) in large quantities at low cost. While hydrofracturing (“fracking”) and other drilling technologies have dramatically reduced the cost of producing such hydrocarbons, the complex and time-varying chemistry of produced hydrocarbons and formation fluids can decrease the production of any given well (or group of wells) and can even cause a well to be shut down for maintenance/re-stimulation, work-overs, or shut-in permanently should conditions deteriorate far enough.

More specifically, such time-varying chemistry presents a number of technical problems. For instance, certain species in the produced fluid can cause corrosion in the well, the wellhead, production equipment, transportation pipelines, gathering facilities, and other points downstream therefrom. Moreover, hydrogen sulfide dissolved (or released) in the produced fluid can present an environmental and/or safety hazard (as well as contributing to some modes of corrosion). Bacteria in the fluid can foul filters, coat sensors, and contribute in their own ways to corrosion. Salts and other chemicals can precipitate out of solution and coat the internal surfaces of various components with scale, thus leading to decreased throughput, inaccurate sensor readings, reduced heat transfer capabilities, etc. Similarly, asphaltenes, paraffin's, hydrates, and/or the like can precipitate from the produced fluid thereby clogging pipelines and/or fouling many types of equipment.

Corrosion, which is often characterized by a loss of metal (or other materials) due to chemical (and/or electrochemical) reactions can eventually degrade and/or destroy structures in the production systems. Corrosion can occur anywhere in these systems, from the bottom of the “hole” (and any tools located therein) up to and including surface-based lines and/or equipment. The corrosion rate(s) will vary with time depending on the particular conditions of the oil field/systems such as the amount of water produced, secondary recovery operations, and pressure, temperature, and/or chemical concentration variations.

Hydrogen sulfide (H2S) presents another problematic chemical/corrosion issue often associated with produced fluids. At low concentrations, H2S has the odor of rotten eggs, but at higher, lethal concentrations, it is odorless. Accordingly, H2S is hazardous to workers with even a few seconds of exposure at relatively undetectable concentrations (by human senses) sometimes being lethal. But even exposure to lower concentrations can also be harmful to personnel with chronic exposure being associated with a number of health issues.

H2S can also cause sulfide-stress-corrosion cracking of metals. Because it is corrosive, the presence of H2S in produced fluids can require costly countermeasure such as using high-quality alloys, stainless steel (and/or other, more exotic materials) for tubing and the like. Such sulfides can be treated chemically, provided that they are detected in a timely fashion. More specifically, the sulfides can be precipitated from water, muds, oils, and/or oil muds by treating them with a sulfide scavenger. Follow up testing with (for instance) a Garrett Gas Train can also be conducted to determine sulfide concentrations in the treated fluids. Moreover tests can indicate the need/desire for caustic soda treatments to raise the fluid's pH and/or the need/desire for zinc-based scavengers to remove sulfides (in the form of ZnS).

Moreover, some produced fluids and/or “muds” host sulfur reducing bacteria (SRBs) and/or other so-called biologics. These anaerobic bacterium (the SRBs) can convert sulfate ions such as SO4-2 into S-2 and HS—, with the concomitant oxidation of a carbon source to H2S. The lignite, lignin, tannins, cellulose, starches, fatty acids, and other organic species found in many produced fluids and/or muds provide carbon based food sources and mineral nutrients for such SRBs. Accordingly, produced fluids can have high (and time-varying) SRB concentrations. Moreover, H2S combined with iron can form iron sulfide, a scale that is very difficult to remove.

SRBs, furthermore, occur naturally in surface waters, including seawater and other potential contamination sources that might be introduced into a well for various purposes (for instance as fracking water). Of course, other biologic species can present corrosion issues as well. Thus, bacteria accumulation can lead to pitting of steel and/or buildups of H2S which increases the corrosiveness of the water (and/or other fluids), thereby increasing the possibility of hydrogen blistering and/or sulfide stress cracking which can result in integrity failures and unintended release of hydrocarbons/produced fluids into the environment.

Before storage of hydrocarbons, muds, fluids, and/or other materials potentially containing SRBs, treatment with a bactericide can inhibit SRB growth. Also, circulating these fluids from time to time, with air injection/entrainment, can retard development of anaerobic conditions which favor the growth of SRBs. In situations in which aerobic biologics are found, blanketing the fluids/muds with an inert gas can retard the growth/propagation of these biologic species but only if they are detected and identified in a timely manner.

Produced fluids can also contain materials which lead to scaling of internal surfaces. Many scales form from mineral salt deposits that may occur in the produced fluids. In many situations, a produced fluid is (or becomes) saturated with certain chemicals during its travel through the various systems disclosed elsewhere herein. More specifically, the fluid might travel from a regime in which the pressures, temperatures, pH, etc. preclude precipitation in any meaningful amount to a regime in which one or more factors have changed leading to saturation conditions and thus precipitation of one or more scale-producing species.

With relatively severe conditions, scale can create a significant flow restriction, or even a plug, in a production system. While scale removal is a common well-intervention operation (with a wide range of mechanical, chemical and scale inhibitor treatment options available), it still introduces labor and consumable costs. Moreover the scale removal additives can affect the chemistry of the produced fluid (for instance altering its pH) which in turn leads to other chemistry related issues (for instance, fostering SRB growth). Additionally, it should be noted that scale-precipitation events are variable in nature, and will typically manifest themselves in a non-static fashion as temperature, pressure, and contaminant concentrations vary over time and in response to discrete events. Again, though, corrective measures depend on timely identification of the potentially problematic species in the comingled fluids.

Asphaltenes paraffins hydrates, and other similar precipitating species can present still other issues for the well operator, owner, and/or other users. For instance, paraffins are hydrocarbon compounds that often precipitate on/in production components as a result of the changing temperatures and pressures within these systems. Heavier paraffins occur as wax-like substances that may build up on internal surface/components and can restrict (or even stop) production flowrates. Paraffins are normally found in the tubing close to surface. Nevertheless, it can form at the perforations of the well casing, or even inside the formation, especially in depleted reservoirs or reservoirs under gas-cycling conditions. Asphaltenes and hydrates present similar issues as those caused by paraffins.

To mitigate these issues, oil well operators and completion companies often introduce certain additives to the fracking water that they intend to pump into a given well. As those skilled in the art know, these operators also add fracking sand or proppant to the fracking water. The sand grains thereof act as “proppants” that increase the degree of fracturing in the well formation thereby leading to increased production in many instances. But these additives often change the properties of the fracking water. Moreover, some of these additives transform the incoming fracking water to a non-Newtonian fluid. Indeed, water/sand mixtures can exhibit non-Newtonian behavior. And of course various properties of non-Newtonian fluids, such as viscosity, can vary with the shear rate in the fluid.

Certain fluids present difficult property-sensing issues. As noted, fracking fluid, which includes water a proppant, can exhibit non-Newtonian behavior in that some or all of its properties vary with shear rate. That is, as such fluids flow through their process systems some properties vary with shear rate and not necessarily (and/or predominately) with pressure, temperature, etc. Accordingly, unless these fluids' properties are measured at the shear rate present in their process systems (and/or points of interest therein), these measurements might be inaccurate.

Furthermore, traditionally, “grab” samples have been used to obtain a relatively small portion of these fluids for analysis in a laboratory or elsewhere. But, these fluids might also exhibit changes in their properties with time. Biological activity, for instance, can drive not only oxygen reduction potential in the fluid, but pH as well. As these properties change, they can drive further property changes in these fluids. Additionally, in many instances, operators of these process systems change chemical dosing of the fluids from time-to-time so grab samples cannot capture the true nature of the fluids at least in a timely fashion. Parrafins, for instance, might begin precipitating from the fluids as pH varies. Thus, while grab samples might provide representative portions of these fluids, many reasons exist that the grab samples will not reflect the real-time, actual properties of these fluids in their respective process systems. For instance, grab samples necessarily fail to reflect shear rates within the process systems and thus almost utterly fail to reflect non-Newtonian conditions.

As noted previously, fracking water represents one non-Newtonian fluid with time-varying properties. Corn starch suspensions, nail polish, whipped cream, ketchup, molasses, syrups, paper pulp in water, latex paint, ice, blood, some silicone oils, some silicone coatings, sand in water, blood plasma, custard, and even water (under certain circumstances) can behave in non-Newtonian manners. Some of these fluids, moreover, will exhibit “shear-thickening.” As noted, sand-water mixtures belong in the category of non-Newtonian fluids. This situation poses problems in the fracking industry because one of the major additives to fracking water is fracking sand.

For decades, fixed-rate chemical application programs have been applied to oilfield fluids with fluctuating chemistries. This approach leads to inefficient chemical dosing and asset management schemes which can adversely impact well production and equipment longevity. In many situations well operators use blenders to treat (that is add additives, sand, etc. into) the incoming fracking water. These blenders often run for long periods of time with/without human users being present to monitor their performance.

It is an object to obtain more accurate measurements of fluid properties and to better control the application of additives to such fluids.

### SUMMARY

The following presents a simplified summary in order to provide a basic understanding of some aspects of the disclosed subject matter. This summary is not an extensive overview of the disclosed subject matter, and is not intended to identify key/critical elements or to delineate the scope of such subject matter. A purpose of the summary is to present some concepts in a simplified form as a prelude to the more detailed disclosure that is presented herein. The current disclosure provides systems, apparatus, methods, etc. for sensing properties of fluids and more particularly for sensing shear-rate dependent properties of fracking water.

Various embodiments provide apparatus, systems, and related hardware-based methods for sensing (non-Newtonian) fluid properties more accurately then heretofore possible. Some embodiments provide platforms which monitor the fluid chemistry and fluid dynamics of hydro-fracturing fluids in real-time; identify the nature/characteristics of that fluid; provide real-time chemistry application and process controls to manage these fluids effectively, and create data streams which can be used to improve fluid management and/or chemistry dosing practices through the stages/phases associated with hydro-fracturing processes.

Briefly, systems of some embodiments comprise suites of integrated analytical sensors (which can include, but are not limited to pH, density, viscosity, temperature, conductivity, oxidation reduction potential (ORP), CO2, dissolved O2 and corrosion index sensors), pressure transducers, temperature transducers, accelerometers, power meters, and flow meters. These sensors communicate with an onboard CPU (central processing unit), PLC (programmable logic controller (PLC), and/or some other processing device of the current embodiment which monitors the fluid characteristics/chemistry/rheology from fluid entering and/or exiting one or more fluid augmentation devices on hydro-fracturing sites (such as fluid blenders, hydration units, pumps, etc.). Additionally, platforms of the current embodiment provide for the monitoring of shear-sensitive and temporal corrosive conditions that could impact production equipment/tubing integrity, and thus provides mechanisms by which these conditions can be mitigated.

As data is collected and analyzed by systems of the current embodiment certain aspects of the monitored process systems are automatically adjusted in real-time to improve the fluid chemistry and/or align them with desired characteristics despite the presence/likelihood of non-Newtonian behavior. For instance, chemical dosing regimes, preventive maintenance regimes, and servicing regimes can be adjusted responsive to the resulting data. Systems of the current embodiment collect data continuously and stream the same to their processors/users which can store, analyze and use that data to develop/implement/control more successful well management practices.

Turning now to a brief discussion of some of the underlying conditions of the process fluids involved, systems of such embodiments include hardware that allows for samples to be run through the sensing system at velocities and shear rates representative of the shear rates observable in the respective process systems (for instance in, or associated with, a fracking water blender). And/or if desired, systems of embodiments can allow for the systematic sampling and holding of samples, so that their characterizes can be observed for configurable durations of time (in accordance with, for instance, ORP declines curved which are based on oxidative demands of the fluid(s)) to characterize the incoming fluid albeit (or, indeed) at static conditions.

Additionally, such sample and hold systems can allow for the sample to be heated to reservoir temperatures and increased pressures, so that the effects of temperature on fluid chemistry parameters and viscosities (and other conditions) can be modeled. Thus, embodiments provide for a more comprehensive determination of down hole conditions than heretofore available. This information is then used to optimize fluid management at the surface, so that the fracking water can be designed to achieve optimal chemistry/dynamics as desired to hopefully stimulate the reservoir in an adequate/as-desired manner. This capability allows for individual and/or local group well completion enhancement practices to be systematically deployed across common geographic areas and similar reservoir formations.

The collection of multiple analytic values as a function of time, stimuli, and fluid response allows for the characterization of down-hole fluid dynamics that can be used to maximize (or at least increase) product yield. This data-driven approach to well management completion deviates significantly from current standard practices, and provides a mechanism to scientifically apply successful chemical and process treatment regimes in the field.

Embodiments provide systems, apparatus, and methods for duplicating a shear rate of a fluid in a process system and for sensing one or more properties at that duplicated shear rate. A system of the current embodiment comprises a port configured to receive a fluid having a shear rate in a process system. It also comprises a valve and a sensor set in communication with the port and a controller in communication with the process system, the sensor set, and the valve. The controller of the current embodiment is configured to receive a sensed flow rate of the fluid from the process system (or the user) and to control the valve so as to duplicate the shear rate of the fluid in the process system. The controller is also configured to sense a property of the fluid at the duplicated shear rate via the sensor set and to output the property of the fluid at the sensor set and at the duplicated shear rate.

If desired, some systems of the current embodiment can further comprise first and second spools. In these embodiments, the sensor set is on the first spool and the system further comprises a pH and ORP sensor on the second spool. In this way the controller can sense the pH and ORP of the fluid and close the valve if the pH is outside of a user selected range. Thus, the controller can sense the ORP of the fluid and close the valve if the oxygen reduction potential is outside of a user selected range. Additionally, or in the alternative, systems of the current embodiment comprise feedback loops in communication with the controller of the process system. Therefore, the sampling controller can send a control signal to the process system controller which is indicative of a control action to take based on the value of the sensed property of the fluid. Furthermore, systems of some embodiments can include a second port, sensor set, and valve. In such systems the first port is in communication with the process system upstream of a blender and the second port is in communication with the process system downstream of the blender. Moreover, the controller of such systems is further configured to sense a second fluid property via the second sensor set and to output an indication thereof. Note that the first and second properties can be of the same type. If desirable, the controller can be configured to determine, and to output a signal indicative of, the difference between the first and second sensed properties.

Systems of the current embodiment can also comprise a solids separator upstream of the second sensor set. And/or in such systems, one or more of the sensor sets can be on a spool with a vertical orientation. As to the sensors, they can be viscosity sensors, corrosion sensors, conductivity sensors, flow meters, turbidity sensors, fluid density sensors, etc. As to the fluid in the process systems, generally, they tend to be non-Newtonian floods such as fracking water, soap, blood, etc.

In accordance with embodiments, methods for duplicating a shear rate of a fluid in a process system and for sensing one or more properties at that duplicated shear rate are provide. One such method comprises various operations such as receiving a fluid via a port and which has a shear rate in a process system. This method (and/or others) also comprises receiving a sensed flow rate of the fluid via a controller and from the process system. Such methods can further comprise controlling a valve with a sampling system controller in communication with the process system controller to duplicate the shear rate. These methods further comprise sensing a property of the fluid at the duplicated shear rate using a sensor set and outputting a signal indicative of the sensed property of the fluid (as measured at the duplicated shear rate).

If desired, methods in accordance with embodiments can operate using a second spool. Such methods further comprising sensing a pH of the fluid using a pH sensor on the second spool and closing the valve if the pH of the fluid is outside of a user selected range. Some methods can comprise sending a signal to the process system controller indicative of a control action to take based on the value of the sensed property of the fluid. In addition, or in the alternative, a second property of the fluid can be sensed by a second sensor set on the second spool. Indeed, in some situation, the first and second sensed conditions can be of the same type. If desired, solids can be separated from the fluid using a solids separator. Moreover, one or more of the sensors can be on a spool oriented vertically.

To the accomplishment of the foregoing and related ends, certain illustrative aspects are described herein in connection with the annexed figures. These aspects are indicative of various non-limiting ways in which the disclosed subject matter may be practiced, all of which are intended to be within the scope of the disclosed subject matter. Other advantages and novel and non-obvious features will become apparent from the following detailed disclosure when considered in conjunction with the figures and are also within the scope of the disclosure.

### BRIEF DESCRIPTION OF THE FIGURES

The detailed description is described with reference to the accompanying figures. In the figures, the left-most digit(s) of a reference number usually identifies the figure in which the reference number first appears. The use of the same reference numbers in different figures usually indicates similar or identical items.

FIG. 1 illustrates a frack water system.

FIG. 2 illustrates shear rates in non-Newtonian fluids.

FIG. 3 illustrates a sensor system for sensing properties of non-Newtonian fluids.

FIG. 4 illustrates a controller for sensing properties of non-Newtonian fluids.

FIG. 5 illustrates a flow chart of a method of sensing properties of non-Newtonian fluids.

FIG. 6 illustrates the flow of fluid relative to the sensor platforms of FIGS. 1 and 3.

### DETAILED DESCRIPTION

The current disclosure provides systems, apparatus, methods, etc. for sensing properties of fluids and more particularly for sensing shear-rate dependent properties of fracking fluid, and processes by which fluid stability over time can be evaluated. Such shear-rate dependent properties include viscosity and density.

Systems of embodiments are designed to measure/monitor process fluid properties in oilfield fluids in real-time. These systems are engineered to maximize the efficiency and longevity of the sensors so that the system can operate for extended periods of time without maintenance/servicing. The sensors onboard these platforms/systems are selected to not only provide accurate data in harsh environments, but to also to provide data that could be used to characterize fluid properties, fluid chemistry, and process trends. In accordance with embodiments, systems allow for (inter alia) the following:
Shear-rate-adjusted fluid properties sensing;
Time-based adjusted fluid properties sensing;
Real-time collection of process variables of interest;
Logging/transmission of variable values to remote database(s);
Identification of potentially problematic conditions within the process fluids and/or associated machinery;
Management of potential problems by user alerts and/or for real-time chemical application or equipment operation.

With regard to fracking fluid, various properties can be accurately measured and monitored during fracking operations. Fracking fluid is a mixture of water and proppant, such as sand. Other chemical additives, such as a biocide, pH adjusters, etc. may be mixed into the fracking fluid. Measuring properties such as viscosity and density, even after a proppant has been added, can be accurately achieved. This is due to measuring these properties at the same shear rate as the fracking fluid flowing in the system. In order to minimize wear on the sensors and to avoid interrupting the flow of fluid while measuring, samples of the fluid are diverted or withdrawn from the system. These samples are then measured. Measurements can be on the flowing fluid or on a held sample of the fluid. A held sample is useful to measure characteristics such as potential biological activity.

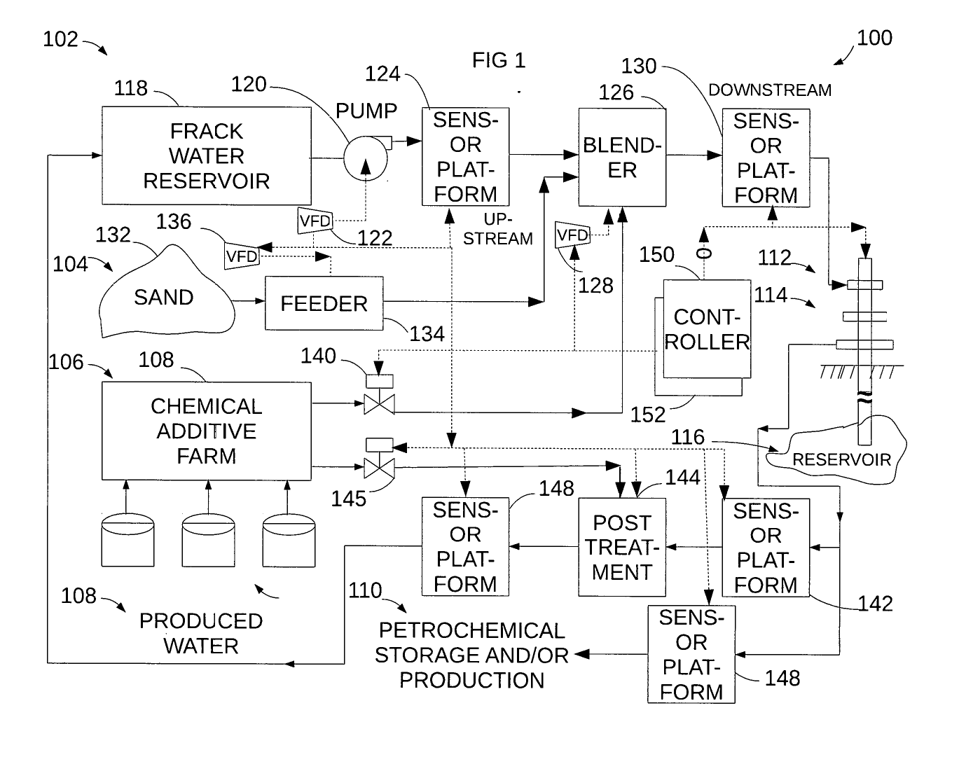

FIG. 1 illustrates a frack water system. The embodiment of FIG. 1 happens to be illustrated in the context of a particular oil well. But it could operate in any number of contexts. Generally, such an oil well is drilled, placed into operation, and thereafter operated to produce various petrochemicals, such as oil, gas, condensates, etc. (hereinafter “oil”). But, for various reasons, an operator/owner or other interested user (hereinafter “user”) might wish to stimulate the oil well. For instance, as the oil well draws oil from the reservoir(s) which it taps, that reservoir can become partially depleted resulting in a lowering of oil production. Or, as increasingly common, the user might want to increase the production via stimulation to increase their economic recovery whether or not a production decline has been observed.

While other types of stimulation exist, fracking will be used to further illustrate aspects of embodiments. In fracking (i.e. hydro-fracturing), the user pumps high pressure, sand-laden water into the well. The water carries the sand to the well and then out into its formation. Once in the formation, the high pressure water fractures the material (typically shale) of the formation causing numerous cracks (i.e. fractures) to propagate through the formation or at least portions thereof. The water also carries the granules of sand into the cracks such that when the pressure is released, the overlying material settles on the granules rather than directly on the lower layer of material from which they were fractured.

The presence of the sand in the water transforms what might otherwise have been a Newtonian fluid (the water) into a non-Newtonian fluid. The non-Newtonian nature of the frack water of course makes at least some of its properties dependent on the shear or shear rate (hereinafter “shear”) instantaneously present at any given location in the frack water. Complicating matters further, the composition of the fluid in the system can vary with time and can also vary as the fluid flows from point to point in the system. Sources of variation are too numerous to enumerate herein. But, for one thing, the reservoirs from which the fluid is drawn change with time. This variation can occur as some chambers/areas of the formation empty and thence the well begins drawing more predominantly from other chambers. Of course, frack water is but one example of a non-Newtonian fluid and the scope of the current disclosure is not limited to frack water but extends at least to any non-Newtonian fluid.

That being said, operators of the well might perform re-work and/or maintenance, on the wells. These operations can (intentionally or otherwise) introduce chemicals into the system. The wells also have something of a life cycle. For instance, during drilling, the fluid flowing from the well will be largely drilling “mud.” But, when the well reaches its production depth, that fluid can begin carrying increasing quantities of hydrocarbons and the species consistent therewith. During completion of the wells, still other chemicals are introduced into the stream. Even when the wells have been placed in operation, stimulation and other work on the well can cause the chemistry of the produced fluid to vary (and often with no warning to those portions of the system downstream from the well).

Also, as the shear, pressures, temperatures, pH, and other characteristics of the fluid change as it moves through the system, various species might precipitate out, off-gas, etc. To make matters still more complicated, the potential presence of so many species in the fluid renders many sensors inoperative, unreliable, imprecise, inaccurate, etc. Thus, operators of heretofore available systems have had to rely on sampling the fluid periodically and manually analyzing it to determine the species in the fluid, their concentrations, their properties, etc. and (hence) the likelihood that one or more problems might be occurring in the system (at, or near, the sample point).

With more particular reference to FIG. 1, the drawing shows a process system 100, a frack water system 102, a sand feed system 104, an additive feed system 106, a produced water system 108, an oil collection system 110, an oil well 112, a Christmas tree 114, a reservoir 116, a frack water reservoir 118, a frack water pump 120, a frack pump VFD (variable frequency drive) 122, an upstream sensor platform 124, a blender 126, a blender VFD 128, a downstream sensor platform 130, a sand source 132, a sand feeder 134, a sand feeder VFD 136, an additive source 138, an additive flow control valve 140, another sensor platform 142, a post treatment system 144, yet more sensor platforms 146 and 148, a platform controller 150, and a process controller 152. For the sake of convenience further aspects of a typical petrochemical production system are not shown. However, those skilled in the art understand that additional upstream, midstream, and downstream processes, systems, equipment, etc. are often involved.

Conditions within the process system 100 can vary widely. For instance, in the reservoirs 116, temperatures can exceed 400 degrees Fahrenheit with pressures reaching 2000 psi or more. Moreover, the fluid in the reservoirs 116 has yet to receive any treatment (at least initially) and has potentially many potentially problematic species in it.

In most situations, these fluids include a mixture of hydrocarbons (unless the wells were drilled to obtain other fluids) and usually some amount of water (often with salts dissolved therein). Needless to say, these conditions often present aggressive corrosion threats. Accordingly, many oil wells 112 comprise downhole instruments measuring the pressure, temperature, flowrate, and other parameters associated with the reservoir 116 (or at least the “bottom” of the oil well 112). Note that process controllers 152 of embodiments can be in communication with these sensors (as well as sensors at other locations in the process system 100) to obtain information therefrom and to take control actions via effectors when conditions warrant.

As disclosed elsewhere herein, moreover, these conditions are not necessarily static. For one thing, as various areas, layers, volumes, formations, etc. associated with the reservoirs 116 are tapped, the varying fluid levels in the reservoirs 116 deliver fluids of varying composition to the oil well 112. At some times, predominantly hydrocarbons might be flowing to the oil well 112 while, at other times, the predominant species might be water. And, at times and/or at particular wells, relatively high volumes of gas (for instance, methane) might be delivered to the oil well 112. Operator activity might also influence the species present in the comingled fluid. For instance, fracking might introduce a large proportion of water into the oil well 112 along with sand and agents which cause the fluid to “gel” thereby enabling the fracking operation. In other situations, operators might believe they have some issue to deal with in the formation. For instance, they might believe that some scale-creating agent has increased in concentration and, therefore they might inject an additive selected to combat that particular scale-related species. In addition, the reservoir characteristics may indeed change over time in developed fields as localized pressures decline, thus creating the potential for localized off-gassing, adiabatic temperature loss and thus precipitation and chemistry shifts. Of course, as these conditions/fluids vary, the fluid properties vary accordingly and in many situations the fluids are non-Newtonian.

As the produced fluid travels toward the surface through the casing of the oil well 112, conditions also change. For one thing, the pressure drops in the casing as the hydrostatic head decreases. The fluid might also cool somewhat as the temperature of the surrounding formation drops (roughly with decreasing depth). The results include a potential effervescence of gases dissolved/entrained in the fluid. Moreover, the changing pressure/temperature point (as well as other changing conditions) might cause other species to precipitate out of solution.

As disclosed elsewhere herein, the additive feed system 106 can also be a source of changing fluid chemistry in the oil well 112. While several additive tanks appear in FIG. 1, it is understand by those skilled in the art that these tanks are representative sources of additives. While there are some situations in which one or more additive tanks might be present near an oil well 112, additives are usually injected into a well (and hence brought to it) on an as-needed basis largely depending on the detected composition of the fluid therein. Nonetheless, these additives are usually injected into the well via the kill wing of the Christmas tree/well head 114 although they could be injected by alternative means such as by other connections at the Christmas tree 114 and of course through the blender 126. That being said, such injections can be unscheduled and can occur without warning to user/operators downstream of the oil well 112.

The Christmas tree 114 of a well allows many operations on the oil well 112 and also represents a point in the process system 100 at which fluid conditions can change. For instance, additives might be injected into the reservoir 116 via the kill wing of the Christmas tree 114. Moreover, the fluid produced by the oil well 112 flows out of the annulus of the casing through the production wing of the Christmas tree 114. More specifically, the production wing often includes a choke through which the produced fluid flows.

The choke is used in many cases to maintain a back pressure in the casing (and reservoir 116) to maintain a relatively constant/controlled flow of the fluid. The choke also, as a result, causes a relatively large delta pressure across itself. Indeed, that delta pressure can be on the order of 800 psi or more. As a result, gas effervescence can occur with attendant fluid temperature decreases (as the gas expands adiabatically across the choke). Accordingly, systems 100 of embodiments instrument the Christmas tree 114 with pressure sensors, temperature sensors, and flow meters which (along with downhole sensors) allow the amount of gas released to be estimated (via the Ideal Gas Equation and/or suitably modified versions thereof).

The choke in many cases is a control valve which basically acts as a variable orifice. And across (from upstream to downstream) the choke shear in the fluid varies rapidly. More specifically, fluid flowing through the center of the orifice might exhibit a relatively constant shear. But, the fluid flowing closer to the edges (of the time-varying orifice) might experience drastic change in shear. At a sufficient distance from the orifice, the shear probably remains more or less static. But as the fluid approaches the orifice, shear probably increases drastically as the orifice chokes the fluid. Plus, as the fluid passes over the edge of the orifice, the shear probably reaches a peak and then rapidly decreases as the fluid leaves the edge of the choke/orifice “plate.”

Of course, the choke is but one of many locations in the system in which the fluid experiences a wide range of shear. For instance, most control valves will cause such shear changes. But, anywhere the system 100 changes the momentum (either velocity, direction, and/or both) of the fluid, the fluid will undergo shear variations. And as the shear varies, the properties of the non-Newtonian fluids in the process system 100 will change accordingly. Thus, at this juncture it might be helpful to consider certain aspects of the embodiment illustrated by FIG. 1.

With ongoing reference to FIG. 1, the process system 100 can typically be divided into several different subsystems. These subsystems include, but are not limited to, the frack water system 102, the sand feed system 104, the additive feed system 106, the produced water system 108, the oil collection system 110, and the oil well 112 itself (along with the reservoir 116).

Perhaps, as the point at which all of the other subsystems converge, the oil well 112 can serve as a handy lead-in point for further disclosures. The oil well 112, on that note, serves to produce oil from the reservoir 116. The reservoir 116 defines an underground void(s) that might be nothing more than a porous structure in some subterranean strata. Often it lies in a layer of shale, limestone, or other porous rock. And by hydro-fracturing that rock, much larger quantities of oil can be economically produced in many cases. Though, hydro-fracturing is not required to practice the scope of the current embodiment.

The Christmas tree 114 serves as a point at which various other subsystems can connect to and support production at the oil well 112. For instance, in the embodiment shown in FIG. 1, the frack water system 102, the sand feed system 104, the additive feed system 106, the produced water system 108, the oil collection system 110 all couple to/communicate with the oil well 112 via the Christmas tree 114 either directly or indirectly. For instance, the frack water system connects there and, through it, so does the sand feed system 104 and the additive feed system 106. Thus, the Christmas tree allows these subsystems to inject frack water, frack sand, and any number of additives into the oil well 112.

On the downstream side of the oil well 112, the produced water system 108 and the oil collection system 110 connect to the oil well 112. While these subsystems are shown separately, it could be the case that the two systems are one and the same with operational circumstances determining whether the produced fluid is treated as water (of some sort), oil, or a combination thereof. Of course, the Christmas tree 114 contains other attachment/coupling points such that fluids/chemicals might be introduced into the oil well independently of the aforementioned subsystems.

With ongoing reference to FIG. 1, the frack water system 102 represents possibly the largest source of additives to be introduced into the oil well 112. The frack water system 102, moreover, pressurizes the water therein and injects it into the oil well 112. Those skilled in the art understand, of course, that fracking even a relatively small reservoir 116 can require literally millions of gallons of water. Many of these wells, moreover, exist in arid/desert environs where water is hard to come by. Much of the available water is often brine which must be treated before its injection. Given these circumstances, an accurate understanding of the frack water chemistry can save a user considerable amounts of money in avoiding adding chemicals that might not be necessary to treat the frack water. And it can help the user dose the correct/desired additive mixture into the oil well 112.

Nonetheless, the frack water system 102 draws water from the frack water reservoir 118 and pressurizes it with the frack water pump 120. That pump can be located as shown in FIG. 1 or can be at other user selected location such as just upstream of the Christmas tree 114. Moreover, at some point, the frack water system 102 often includes a blender 126. One thing the blender 126 does is to take the sand and additives from their respective subsystems and blend them with the frack water.

Again, the volumes of materials involved might surprise the uninitiated. To lend some perspective to it, though, consider that fracking an oil well 112 can require literally a train load of frack sand. Corresponding quantities of additives are also involved and often added via the blender 126. Blenders 126 come in a variety of configurations but one can think of a blender 126 as being a large mixing tank with a paddle of sorts stirring the materials therein into a relatively homogeneous mixture, and if not then into a suspension.

As to the sand feed system 104 illustrated by FIG. 1, it begins with a sand source 132. That source can be nothing fancier than a gigantic pile of sand. Although, in many cases, a depot will exist into which the sand trains pull and offload their cargo through undercarriage gates. Screw feeders typically push the received sand forward through the system and into the blender 126. Of course other types of feed arrangements are within the scope of the current embodiment. Though it might be worth noting that the sand involved can be selected based on its average grain size (or distribution of sizes) and typical shape. Indeed, there is one preferred location for obtaining such sand located in Canada.

The additive feed system 106 of the embodiment illustrated by FIG. 1 might bear a few words too. Typically it includes a number of tanks and/or tankers storing the various additives. Although, as mentioned elsewhere, additives can be injected directly into the oil well 112 via the Christmas tree thereby bypassing the additive feed system 106. The additive feed system 106 combines these additives into, hopefully, a blend representing the mixture of chemicals which the user wishes to inject into the oil well.

The additives involved come in a large variety. For instance, oil well “mud” can be a large constituent of the additives, particularly during well drilling and finalization. pH buffers, biocides, anti-scalants, etc. can all be included in the fluid (and/or solids) passed to the blender 126 from the additive feed system 106. The flow of the additives is controlled by the additive flow control valve (FCV) 140 so that these additives can be metered into the frack water via the blender 126. See FIG. 1. Note that the additive feed system 106 can also supply various additives to the produced water and/or oil as illustrated by the post treatment system 144 and associated FCV 145.

Still with reference to FIG. 1, a few words might be in order regarding the produced water system 108. As noted elsewhere herein, the oil well 112 is likely to produce a mixture of produced water and oil. Especially, during well drilling and/or completion a relatively large proportion of the fluid produced from the well can be expected to be water (albeit with potentially large fractions of other species entrained therein) with some oil also potentially being present. As the well moves into production, more and more of the produced fluid typically becomes oil. Regardless, the produced “water” can be separated from the produced fluid by a separator (not shown) and bled off for subsequent reuse via the produced water system 108. The oil, having been separated from the produced fluid too, can be drawn off via the oil collection system 110 for subsequent storage, distribution, refilling, use, etc.

To reuse the water though often requires that it be treated. And so many process systems 100 (such as that shown in FIG. 1) include some type of post treatment system 144. Again, additives are blended into the produced water via such devices so that the water is suitable for reuse in the oil well 112. And the post treatment system 144 can be a blender of sorts fed by a FCV 145 as illustrated by FIG. 1. The oil produced by the oil well 112, meanwhile, can be drawn off in the oil collection system 110 and treated as the user desires. Note that many of the aforementioned subsystems employ analog control devices such as the various FCVs as well as various motors driven by variable frequency drives (VFDs) 122, 128, and 136. Although other types of analog control devices are within the scope of the current disclosure as are discrete control devices. These control devices allow the process controller 152 to regulate the process system 100. As is further disclosed herein, the platform controller 150 can provide feedback signals to the process controller 150 such that the process controller can adjust the operation of the process system responsive to real-time data regarding shear-dependent properties of the process fluid.

As alluded to elsewhere herein, efficient and reliable operation of the process system 100 can be enhanced should the user (and or controllers 152 of the process system 100) have accurate and precise information regarding the properties of the various fluids in process system 100. The properties of many of theses fluids/mixtures/suspensions can vary with shear rate when the resulting fluid is a non-Newtonian fluid. To obtain real-time, reliable, accurate, and precise measurements of these properties, the embodiment illustrated by FIG. 1 includes a number of sensor platforms 124, 130, 142, and 148 which can capture accurate data regarding shear dependent properties. Although more/fewer sensor platforms could be employed. And note that while FIG. 1 shows one platform controller 150 and one process controller 152, their operations could be distributed across various controllers. Indeed, in embodiments, the platforms each have their own onboard controller. All of which can be networked together. Alternatively, a single controller could be used as both a platform and process controller.

In the embodiment of FIG. 1, one pair of sensor platforms 124 and 130 bracket the blender 126. Another pair of sensor platforms 142 and 148 bracket the post treatment system 144. In each situation, the paired sensor platforms can monitor the incoming and outgoing properties of the fluids as they traverse the respective process equipment. And, by comparing the before and after conditions at each location, the effectiveness of the processes involved can be evaluated. Moreover, corrective actions can be taken whether manually, automatically, or a combination thereof if the change in properties suggest that a chemistry-related change might be in order for the fluid involved. But for these results to occur, accurate sensing of the fluid's properties is helpful and shear-dependent properties pose a heretofore unsolved challenge in this regard.

Note also that the process controller 152 serves to control many if not all of the active control elements in process system 100. It does so through many analog and/or digital actuators. And for purposes of illustration, FIG. 1 shows a number of VFDs 122, 128, and 136 which convert an analog signal (usually 4-20 mAmp) into an output speed for driving some motor. Similarly, the process controller drives the actuators of FCVs 140 and 145 to control certain aspects of the process system 100.

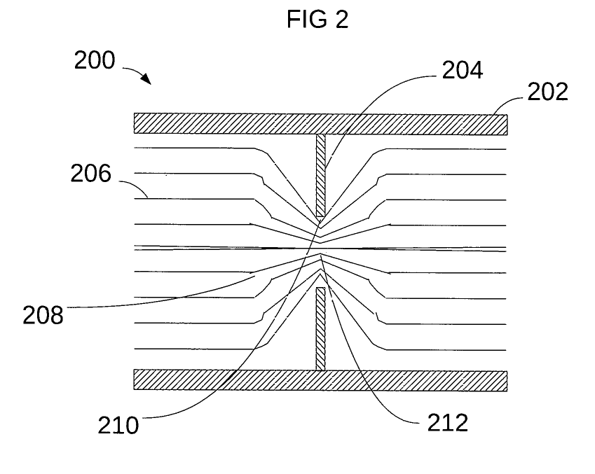

It might now be helpful to consider a typical scenario in which time and location varying shear might be present. Thus, FIG. 2 illustrates shear rates in non-Newtonian fluids. For illustrative purposes FIG. 2 shows a cross-section of an orifice 200 along with a number of flow lines depicting movement of fluid through the orifice 200. While FIG. 2 shows an orifice, it is understood by those skilled in the art that any restriction within a process system will cause analogous shear changes as those disclosed in this illustrative scenario. Indeed any control valve, choke, inlet/exit port, pump blade, sensor probe, sensor cavity, etc. is likely to cause varying shear at least in a localized environment. Indeed, even surface roughness in a pipe (or reservoir) can cause localized shear variations in the boundary layer of the process fluid. Of course, shear variations can and do occur on large, for instance the flow rate in a given section of a process system 100 might change.

Or, the fluid might encounter a bend or elbow in the system and of course mixers and the like will cause shear variations: both local/micro, large scale, constant, and/or time varying in various combinations. And since fluid properties vary with shear in non-Newtonian fluids, these variations present environments in which adverse events can occur within the fluid and/or at its points of impingement/contact with the vessels, pipes, sensors, etc. of process system 100. For instance, some species might precipitate within one or more of these (micro) environments leading to scaling, clogging, fouling, etc. issues. These micro events can, potentially, grow/morph into larger scale issues with time.

With reference still to FIG. 2, the drawing illustrates one scenario in which shear varies. FIG. 2 shows an orifice 200 in a process pipe 202. The orifice 200 of course includes a plate 204 with an opening in the middle through which the process fluid passes. In the far field 206, the shear rate is constant as a function of time as long as the flow rate remains the same. Though it is noted here that the fluid can experience more shear near the walls of the pipe 202 since the fluid will tend to drag across that surface until at some distance the boundary layer with the wall fades and the fluid flows without substantial interaction with the wall. Downstream, closer to the orifice 200, a near field develops in which the fluid begins to react to the presence of the orifice 200 in the pipe 202. In essence the flow “pinches down” as it approaches the orifice. Near the center of the pipe 202 in the near field 208, the fluid velocity and shear rate might remain more or less constant—the same as the shear rate upstream and near the center of the pipe 20. But, as the flow lines of FIG. 2 illustrate, shear rate varies with location both radially and longitudinally.

As the fluid flows passed the edge 210 of the orifice 200, it necessarily accelerates (both longitudinally and radially) causing shear rate variations both radially and longitudinally. Within the aperture of the orifice 200, of course, fluid velocity is likely to be at its maximum (assuming the fluid is more or less incompressible, with compressible fluids presenting further shear variations). And the shear across that aperture is likely to vary widely as a function of radius. Thus, a wide variety of shear environments exist in even a simple situation such as that presented by the orifice 200. Should the orifice size change (as with a control valve)—or not be radially symmetric (as with many orifice-like restrictions such as ball and/or gate valves) further more complex shear variations will occur with which those skilled in the art are familiar.

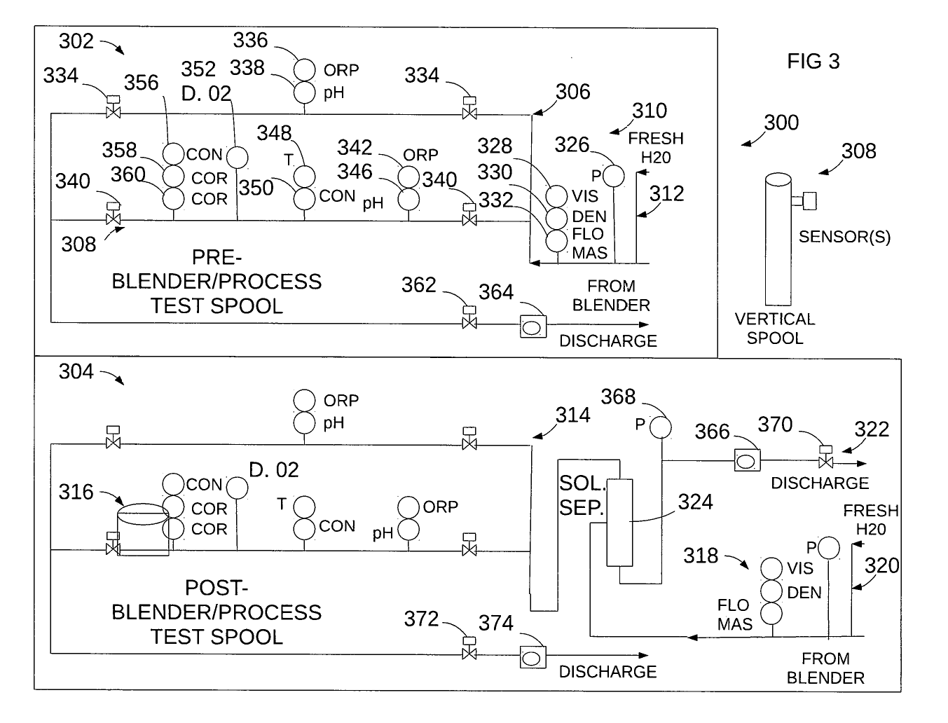

With continuing reference to FIG. 2, it might be the situation that bulk conditions in the fluid would present a rather benign environment. In such environments species precipitation would not be expected. Nor would (in a well designed system) corrosion, scaling, fouling, etc. But, given the shear variations in many local/micro environments, conditions might give rise to localized issues. And these micro issues could evolve into much larger ones. For instance, should conditions near the edge 210 of the orifice allow a species to precipitate, it could be the case that the orifice 200 becomes coated with the precipitant at a particular location on the orifice plate. And the resulting deposit could grow to plug the orifice. Many users would likely not be expecting such a result since bulk conditions appear to be favorable. Indeed, the Inventor suspects that many “unsolvable,” “unpredictable,” “vexatious,” etc. issues in the process industry might arise from the interplay between bulk and micro (shear-dependent) conditions. Thus as will be disclosed further with reference to FIG. 3, embodiments seek to duplicate the shear environment in given process systems and to measure fluid properties under such conditions.

FIG. 3 illustrates a sensor system for sensing properties of non-Newtonian fluids. More particularly FIG. 3 illustrates an overall platform 300, an upstream platform 302 (therein), a downstream platform 304, a catch and hold spool 306, an analytics spool 308, an initial conditions spool 310, a fresh water inlet 312, another catch and hold spool 314, another analytics spool 316, another initial conditions spool 318, a separated solids spool 322, and a solids separator 324.

Generally, the platform 300 includes the two platforms 302 and 304 that could be stand alone platforms/systems of their own. However, the Inventor has found that it can often be helpful to monitor before and after conditions associated with some change in a particular process, some particular piece of process equipment, a particular point in the process system 100 under investigation etc. Thus, the embodiment illustrated by FIG. 3 shows the two platforms 302 and 304 as being parts of an integrated package configured for before/after monitoring situations. The upstream platform 302 can be plumbed into a process system 100 upstream of some location at which fluid property changes are likely, expected, suspected, etc. The downstream platform 304 can be plumbed in downstream of that location. Thus, the platform 300 can capture before/after data and in a shear-sensitive manner.

The downstream platform 304 in some aspects mirrors the upstream platform 302 so for now, the disclosure will focus on the upstream platform 302 and will also disclose pertinent differences between the two platforms 302 and 304. That being said, the upstream platform 302 comprises the two spools 306 and 308 as well as the initial conditions spool 310. One function of the initial conditions spool 310 is to characterize the incoming fluid (at a bulk level) with/without consideration being given to potential shear rates in the process/system 100. In other words, the initial conditions spool 310, in part, can mimic traditional fluid property measuring techniques. Though, if it shares a common internal diameter and surface roughness with that of the analytics spool 308, its readings can also be shear adjusted.

With regard to the catch and hold spool 306, one of its functions is to (on a periodic, as desired, etc. basis) catch a sample of the incoming process fluid and hold it. While the catch and hold spool 306 holds the fluid sample, it monitors various properties of the fluid to 1) confirm that the pH of the fluid is benign to the analytics spool 308 (and/or other portions of the platform 300) and to 2) detect whether certain changes might be occurring in the fluid that could lead to adverse conditions for other portions of the platform 300. And if either the fluid is potentially adverse or could become adverse to other portions of the platform 300, the platform controller 150 can be configured to isolate platform 300 from the overall larger process system 100.

One purpose of the analytics spool 308 is to duplicate a selected shear as it is thought to exist somewhere in the process system 100 (see FIG. 1). Another purpose is to sense fluid properties at that shear rate. Accordingly, the analytics spool 308 comprises and/or relies on some type of flow control element. The spool is also in communication with the process controller 152 so that it can set the desired shear (rate)/fluid velocity proportionally to the flow rate sensed by the process controller 152 (or otherwise made available to the platform controller 150). Similar considerations apply to the spools of the downstream platform 304.

The downstream platform 304 also includes the solids separator 324 and associated separated solids spool 322. In some instances the platform 300 can be plumbed to detect conditions before and after the introduction of solids (whether intentional or not) into the process fluid. For instance, in a fracking environment, a blender 126 will mix sand into the fracking water. And those sand particles can adversely affect a number of relatively sensitive sensors in the analytics spool 316 as well as other equipment. Thus, platform 300 includes the solids separator positioned upstream of the analytics spool 316 to protect the same from damage (primarily, but not limited to, abrasion) by the sand particles as the fluid flows therein.

With continued reference to FIG. 3, it might now be helpful to consider the various components of the upstream platform 302. These components, in the current embodiment, include (but are not limited to): a pressure sensor 326, a viscosity sensor 328, a density sensor 330, a flow meter 332, isolation valves 334, an oxygen reduction potential (ORP) sensor 336, a pH sensor 338, isolation valves 340, an ORP sensor 342, a pH sensor 346, a temperature sensor 348, a conductivity sensor 350, a dissolved oxygen sensor 352, a conductivity sensor 356, a corrosion sensor 358, a corrosion sensor 360, and a flow control valve 362.

The flow meter 332 provides feedback to the flow control valve 362 so that, in conjunction with the controller 150, the platform 300 can hold its flow at a desired setpoint (or if not constant, at the desired variable rate). Of course that setpoint can be provided on a continuous by the platform controller 150.

Also, the user can choose that setpoint based on duplicating a shear rate in the process system 100 of interest. For instance, suppose that a particular piece of equipment in the process system 100 is experiencing fouling that cannot be adequately explained in accordance with heretofore available troubleshooting practices. The user can, from information available from/about the process system 100 (i.e. a flowrate through that piece of equipment and its geometry), set a flow rate through the platform 300 (adjusted for the geometry of the platform 300) that should (in the analytics spool 308) duplicate the shear rate in that piece of equipment. Thus, the sensors in that spool will sense the fluid properties at/near the shear rate in that piece of equipment. Accordingly, even if the fluid is non-Newtonian, an accurate understanding of the fluid properties in the equipment can be obtained. This information, in turn, should allow for a better understanding of why that equipment is experiencing some issue. Of course, the platform controller 150 can receive a real-time signal from the process system 100 indicative of the flow rate therein and adjust flow control valve 362 accordingly (using feedback provided by flow meter 332).

Still with reference to FIG. 3, the viscosity sensor 328 and density sensor 330 begin the characterization of the incoming fluid at the selected flow rate/shear. Note that the initial conditions spool 310 is sized the same as the analytics spool 308 in embodiments. That is, they have a common internal diameter, internal surface roughness, etc. and it is this geometry which the user can consider when selecting a flow rate (and corresponding/desired shear environment). Thus, the sensors in both spools 308 and 310 sense fluid properties at the selected flow rate/shear.

The initial conditions spool 318 can include a temperature sensor as well as the pressure sensor 326. Indeed, in some embodiments, it might be desirable to heat the fluid to a temperature similar to that at the point of interest in the process system 100. Thus, the initial conditions spool 318 could include a fluid heater, or for that matter a fluid cooler if desired. Thus, if some property with shear-dependent or not is also temperature dependent, the platform 300 can adjust conditions accordingly prior to the sensor set of platform 300.

With continuing reference to FIG. 3, it might now be helpful to consider the catch and hold spool 314 in additional detail. The catch and hold spool 314 allows the system to capture a sample of the fluid and hold it for evaluation. More particularly, at least two potential conditions in the incoming fluid might merit some evaluation. For instance, the pH of some process fluids can be expected to be either quite high (basic) or quite low (acidic). And it might be the case that the platform 300 (or analytics spool 316) might include components sensitive to either condition.

Another issue that the catch and hold spool 306 can help address is the potential presence of biological species in the process fluid. Hydrocarbons (i.e. oil) are rich in carbon and often carry biological species. If these species are allowed to proliferate they can coat the interior surface of process equipment with films of organic material. This “slime,” if you will, can foul sensors, reduce throughput, reduce heat transfer efficiency, etc. Thus, their detection can be beneficial to the operation of platform 300 in particular and process system 100 more generally. The catch and hold spool 306 includes the ORP sensor 336 to assist in identifying the potential presence of biological species in the fluid.

Thus, from time to time (on a periodic or other basis), the platform controller 150 can close the isolation valves 334 and hold the fluid there between. If the pH sensor reveals a pH which is either too low or too high (based on user selected thresholds), the platform controller 150 can also close isolation valves 340 to prevent potentially corrosive fluid from entering the analytics spool 308.

The platform controller 150 can also determine whether biological activity might be occurring in the fluid. By holding the fluid between the isolation valves 334, the platform controller 150 can allow these species time to metabolize carbon bearing material in the fluid. As they do so, if present, the ORP sensor 336 should reveal a change indicative of such biological activity. And, if desired, the platform controller 150 can signal the user that the (increased) application of a biocide might be warranted. In the alternative, or in addition, the platform controller 150 can close the isolation valves 340 to prevent fouling of the sensors in the analytics spool 308. Thus, the catch and hold spool 306 can provide certain safeguards to the operation of the platform 300.

Presuming that the platform controller 150 has left the isolation valves 340 open, the remainder of the sensor set of the upstream platform 302 can characterize the process fluid. For instance, the ORP and pH sensors 342 and 346 respectively allow the acid/base nature of the fluid and the degree to which it might contain biological species to be identified. Note that these sensors operate at the selected flow rate/shear as disclosed elsewhere herein. They also operate continuously (as does the rest of the analytics spool 308) even while the catch and hold spool 306 has isolated a sample of the fluid therein.

The analytics spool 308 also includes the conductivity sensor 350 and associated temperature sensor 348. Thus, the platform 300 can sense the conductivity and likely salinity of the fluid (with both temperature and shear being accounted for). Likewise, the dissolved oxygen sensor 352 allows the fluid to be characterized with regard to the presence of dissolved oxygen (again with temperature and shear being accounted for as might be desired). Further still, the corrosion sensors 358 and 360 allow the fluid to be further characterized as to conductivity and corrosion (with shear being accounted for).

Still with reference to FIG. 3, at this juncture a discussion of the downstream platform 304 might be helpful. Much of the downstream platform 304 mirrors the upstream platform 302 and no further comment will be made in that regard for the sake of brevity. However, there are some differences between the upstream and downstream platforms 302 and 304 respectively.

For one thing, and while not limiting, the downstream platform 304 is often plumbed into the process system downstream of some point of interest while the upstream platform 302 is often plumbed into the process system 100 upstream of that point of interest. Though, the two systems 302 and 304 could be plumbed into completely different process systems 100 independent of one another if desired without departing from the scope of the current disclosure.

Also, the downstream platform 304 includes the solids separator 324 and associated separated solids spool 322. These components allow the downstream platform 304 to be plumbed into process systems at points with relatively heavy solids loading. For instance, the downstream platform 304 can be plumbed in downstream of the blender 126 (see FIG. 1) in which significant quantities of sand are introduced into the process system 100. The solids separator 324, which is optional, is typically a cyclone separator although any type of solids separator could be used. Moreover, the separated solids spool 322 includes a sight glass 366 which allows users to visually observe the fluid/solids mixture-suspension as it flows through this spool. It also includes pressure sensor 368 and flow control valve 370 such that as pressure builds in the separated solids spool 322, the platform controller 150 can discharge the contents thereof back to the blender 126 or else where. Note that the solids separator, being positioned upstream of the sensor set in the downstream platform 304 can greatly reduce the presence of solids in the fluid flowing passed the sensors therein. This action, of course, can serve to prolong the service life of these instruments and, more particularly, the sensors most susceptible to wear due to solids impingement thereon.

As an alternative to the solids separator, the inside diameter of the spools 314, 316 can be increased to slow the fluid velocities across sensitive instruments, such as the sensors. After the fluid passes the sensors, the fluid velocities are increased by reducing the inside diameter.

Moreover, one or both analytics spools 308 and/or 316 can be oriented vertically. This vertical orientation helps prevent sedimentation from occurring in the respective spools. Indeed, the fluid velocity can keep the solids therein entrained as the fluid flows up/down and then the solids are swept out of platform 300 by the fluid. And the fresh water inlet 312 can be used to cleanse the upstream platform 302 with fresh water, detergents, solvents, and/or a combination thereof if desired.

Still with reference to FIG. 3, both the upstream and downstream platform 302 and 304 include return legs through which the fluid returns to the process. Of course the fluid can be disposed of otherwise without departing from the scope of the disclosure. These return legs include flow control valves 362 and 374 respectively thereby allowing the platform controller 150 to maintain the selected flow rate/shear through each platform 302 and/or 304. They also includes sight glasses 364 and 374 and other sensors to allow users to evaluate conditions in these return legs.

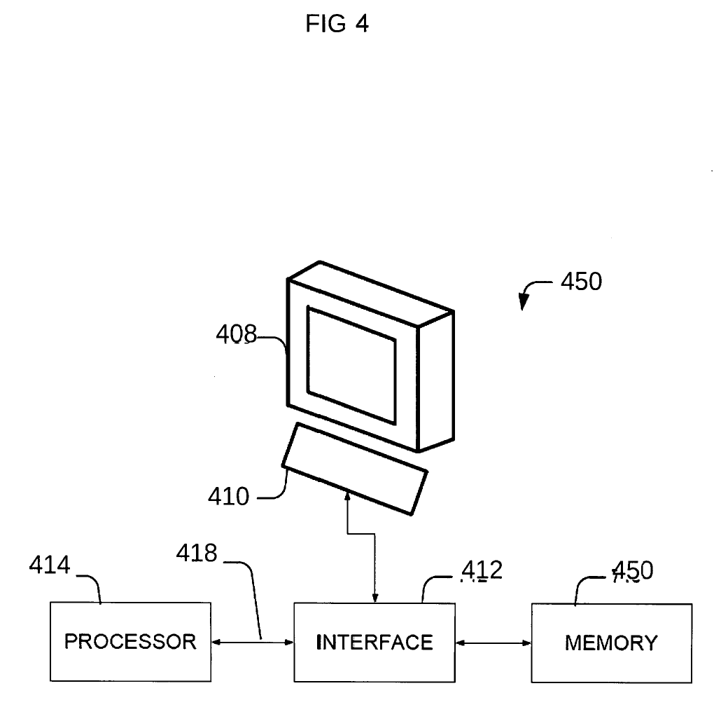

FIG. 4 illustrates a controller for sensing properties of non-Newtonian fluids. A few words might be in order about the controller(s) 450 and/or other systems, apparatus, etc. used to control systems and/or perform methods in accordance with various embodiments. The type of controller 450 used for such purposes does not limit the scope of the disclosure but certainly includes those now known as well as those which will arise in the future. But usually, these controllers 450 will include some type of display 408, keyboard 410, interface 412, processor 414, memory 416, and bus 418. Nonetheless, these computers, when used as a controller 450 for systems/methods of embodiments are specially programmed to do so rather than being mere generic computers.

That being said, any type of human-machine interface (as illustrated by display 408 and keyboard 410) will do so long as it allows some or all of the human interactions with the controller 450 as disclosed elsewhere herein. Similarly, the interface 412 can be a network interface card (NIC), a WiFi transceiver, an Ethernet interface, cell connection, etc. allowing various components of controller 450 to communicate with each other and/or other devices. The controller 450, though, could be a stand-alone device without departing from the scope of the current disclosure.

Moreover, while FIG. 4 illustrates that the controller 450 includes a processor 414, the controller 450 might include some other type of device for performing methods disclosed herein. For instance, the controller 450 could include a microprocessor, an ASIC (Application Specific Integrated Circuit), a RISC (Reduced Instruction Set IC), a neural network, etc. instead of, or in addition, to the processor 414. Thus, the device used to perform the methods disclosed herein is not limiting.

Again with reference to FIG. 4, the memory 416 can be any type of memory currently available or that might arise in the future. For instance, the memory 416 could be a hard drive, a ROM (Read Only Memory), a RAM (Random Access Memory), flash memory, a CD (Compact Disc), etc. or a combination thereof. No matter its form, in the current embodiment, the memory 416 stores instructions which enable the processor 414 (or other device) to perform at least some of the methods disclosed herein as well as (perhaps) others. The memory 416 of the current embodiment also stores data pertaining to such methods, user inputs thereto, outputs thereof, etc. At least some of the various components of the controller 450 can communicate over any type of bus 418 enabling their operations in some or all of the methods disclosed herein. Such buses include, without limitation, SCSI (Small Computer System Interface), ISA (Industry Standard Architecture), EISA (Extended Industry Standard Architecture), etc., buses or a combination thereof.

More specifically, the controller 450 can be connected to the following instruments and controls: the solids separator 324 (if it includes actively controlled components), the pressure sensor 326, the viscosity sensor 328, the density sensor 330, the flow meter 332, the isolation valves 334, the ORP sensor 336, the pH sensor 338, the isolation valves 340, the ORP sensor 342, the pH sensor 346, the temperature sensor 348, the conductivity sensor 350, the dissolved oxygen sensor 352, the conductivity sensor 356, the corrosion sensor 358, the corrosion sensor 360, the flow control valve 362, their counterparts in the downstream platform 304, the pressure sensor 368, and flow control valve 370. And in some embodiments, the controller 450 communicates with a process controller 452 through the interface 412 and/or otherwise. The process controller 452, of course, communicates with various sensors and/or effectors to control the larger process system 100 (see FIG. 1). Note that the combination of hardware communications and the methods executed by the processor transform the controller 450 from a generic computer into a specially programmed computer creating non-abstract transformations in the real word (for instance, specific control actions changing the composition of the process fluid).

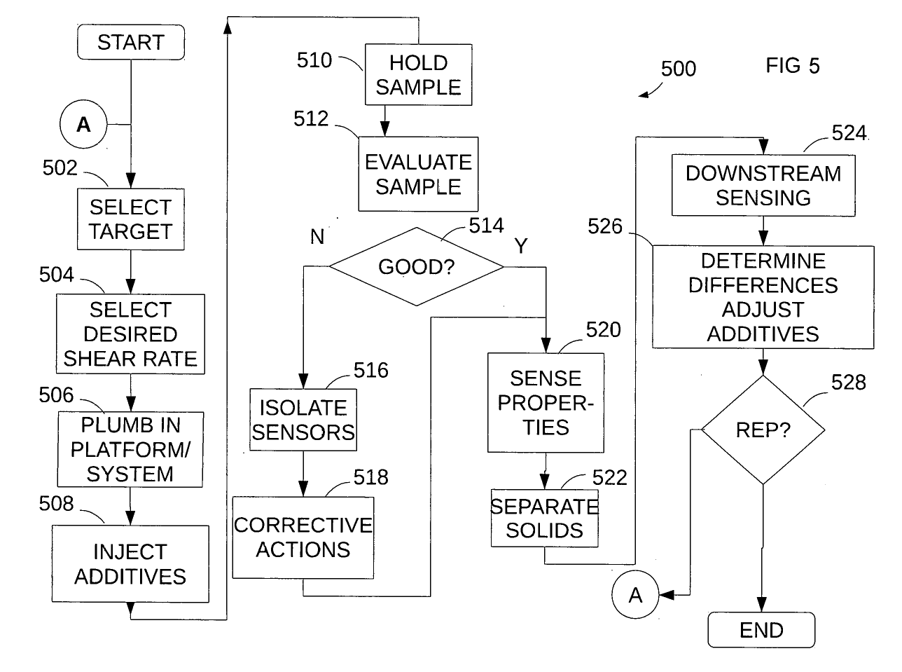

FIG. 5 illustrates a flow chart of a method of sensing properties of non-Newtonian fluids. The method 500 of FIG. 5 includes various operations such as operation 502 in which a user can select a process, piece of process equipment, or some other object for which information might be sought regarding the fluid properties thereof. The blender 126 of FIG. 1 could be one such piece of equipment. The post treatment system 144 could also be a candidate as many other processes can be monitored. Of course, in accordance with embodiments, the fluid can be a non-Newtonian fluid. Although method 500 can be performed for Newtonian fluids as well.

The user can then determine the shear rate which they wish to duplicate in the platform 300. For instance, a pipe exiting the blender 126 would have a certain internal diameter and surface roughness. Additionally, it is likely that the process system of which the blender 126 is a component would have either a flow rate or, more likely, a variable flow rate and the blender 126 would likely be instrumented with a flow meter at it's discharge. Thus, the process flow rate would be known whether it is constant of variable. Moreover, knowing the internal diameter and surface roughness of the analytics spool 308 and/or initial conditions spool 310, the user could calculate a flow rate for the platform 300 which would likely duplicate the shear at the point of interest in the process system 100. See 504.

Having selected a piece of equipment to monitor and determined the desired flow rate/shear environment, the user can plumb in the upstream platform 302 and downstream platform 304. Of course, the user can do so in a manner allowing these systems 302 and 304 to “straddle” or “bracket” the selected piece of equipment. The platform 300 can therefore obtain before and after data concerning how that piece of equipment is affecting the process fluid and/or its properties. See 506.

At 508, if the process system 100 is not already operating, it can be turned on. Or, if it is operating, whatever isolation valves that might have been used while plumbing platform 300 to it can be opened. And, of course, one way or another, additives can be injected into the process fluid. For instance, sand can be injected into fracking water via the blender 126. Additional/alternative materials can be injected into the process fluid. Theses additives change the nature of the process fluid, of course, and can turn even a nominally Newtonian fluid (such as water) into a non-Newtonian fluid. As a result, the properties thereof become (or were and/or are) shear-dependent. Moreover, the addition of additives might affect properties associated with some other additive. For instance, adding a base/acid buffer to the process fluid can, potentially, increase the likelihood of microbial growth in the fluid and hence H2S. See 508.

At 510, system 500 can grab and hold a sample via grab and hold spool 306 for evaluation. For instance, the platform controller 150 can use pH sensor 338 to determine the pH of the incoming fluid. And if the pH falls outside of a user selected range, the platform controller 150 can alert the user and/or signal the process controller 152 so that that controller can adjust the process system 100 if desired. Moreover, with the isolation valves 334 closed, any microbes in the process fluid can continue metabolizing carbon bearing material in the process fluid. As a result, and over time, the ORP in the process fluid will change reflecting these metabolic processes. Readings from ORP sensor 336 will likely reflect those changes and the platform controller 150 can determine such (see 514). If the ORP lies, or comes to lie outside of a user selected range, the platform controller 150 can take appropriate actions. See 514.

For instance, the platform controller 150 can close isolation valves 340 to isolate the analytics spool 308 from the fluid with out-of-range pH and/or ORP as indicated at 516. Moreover, the platform controller 150 can signal the user/process controller 152 that an adjustment to the additive regime might be desirable. Moreover, the platform controller 150 can provide the process controller 152 with analog signals for the sensed pH and/or ORP in the sample. Accordingly, process controller 152 can make adjustments to the same as indicated at 518.

If, though, the fluid is within both pH and ORP ranges, the platform controller 150 can begin sensing the various properties for which analytics spool 308 includes sensors. Of course, once either pH or ORP (if out of bounds) return to their respective user selected ranges, the platform controller 150 can open the isolation valves 340 for the analytics spool 308 and sense the various fluid properties (and at the selected shear rate). Reference 520 indicates such property sensing.

While the current disclosure has focused on the upstream platform 302, method 500 can also include similar operations associated with the downstream platform 304 and its sensors. Of course, if the solids loading in the fluid (post-blender 126), are deemed to be high enough these solids can be removed (at least in part) by the solids separator 324. The sensing via the downstream platform 304 can take place with/without solids separation as might be desired. See reference 524.

With continuing reference to FIG. 5, the platform controller 150 can make comparisons between like properties as sensed by the upstream platform 302 and the downstream platform 304. The platform controller 150 can indicate to the user and/or the process controller 152 the changes in these properties across the monitored piece of equipment. And, if desired, either the user or the process controller 152 can adjust the additive regime should the sensed differences be deemed insufficient or otherwise not as desired. See reference 526.

Reference 528, moreover, indicates that method 500 can be repeated. Furthermore, it can be repeated in whole, or in part, as might be desired. Otherwise, method 500 can end.

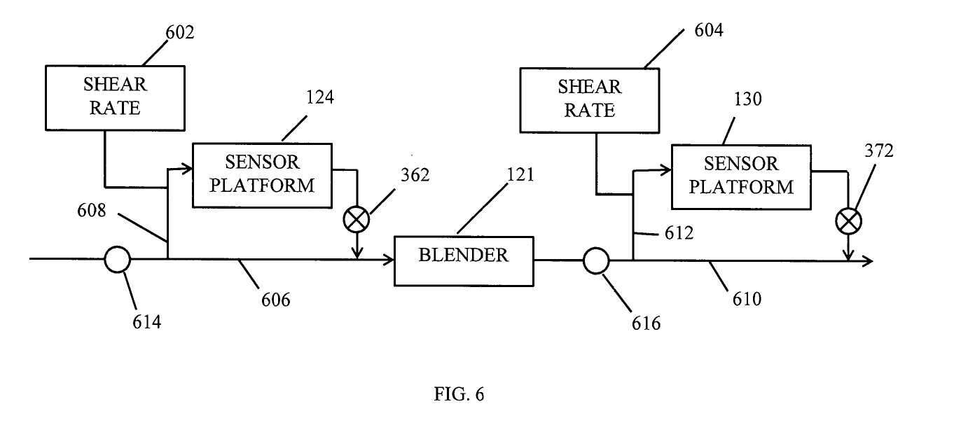

FIG. 6 shows the flow of fluid relative to the blender 121 and the sensor platforms 124, 130 of FIGS. 1 and 3. Fluid flows into the blender 121 through a main inlet line 606. In the blender 121, proppant and other additives are added to the fluid. Fluid then exits the blender in a main outlet line 610. An inlet flow meter 314 is provided in the main inlet line 303. Likewise, an outlet flow meter 616 is provided in the main outlet line 610. The flow meters 614, 616 measure the volume of fluid flowing in the respective lines. The inside diameters of the lines are known, allowing the fluid velocity and rate to be determined on the inlet and outlet sides.

On the inlet side, a portion of the fluid is diverted from the main inlet line 606 into a secondary inlet line 608. One or more shear rate sensors 602 measure the shear rate of the fluid in the secondary inlet line 608. These sensors 602 include temperature, kinematic viscosity, dynamic viscosity, mass and density. This fluid flows into the sensor platform 124, discussed in more detail above. The flow of fluid through the secondary inlet line 608 is controlled by the valve 362. The valve 362 controls the flow so that the shear rate of fluid in the secondary inlet line 608 is the same as (within predetermined tolerances) as the shear rate of fluid in the main inlet line 606. Thus, the measurements on the fluid in the sensor platform 124 are taken at the same shear rate as the fluid in the main line 606. (Note that the fluid in the catch and hold spool 306 is not flowing when the fluid sample is held.) The fluid from the sensor platform 124 is returned to the main line 606.

On the outlet side, the setup is the same as on the inlet side, with a secondary outlet line 612 diverting fluid flow from the main line 610 to one or more shear rate sensors 604, into the sensor platform 130, through the valve 372 and back to the main line 610.

The current disclosure provides embodiments for sensing shear-dependent fluid properties. Various embodiments include means for duplicating a selected shear rate in a process system and measuring these properties in the duplicated shear environment. Thus, additive regimes can be adjusted and operated in real-time and in more efficient, effective manners. Furthermore, many issues such as scaling, corrosion, H2S presence, etc. can be mitigated and can be mitigated automatically despite the presence of shear dependent properties of the process fluid. Systems, apparatus, and methods of embodiments therefore provide for more reliable, cost-effective process system operations.

Although the subject matter has been disclosed in language specific to structural features and/or methodological acts, it is to be understood that the subject matter defined in the appended claims is not necessarily limited to the specific features or acts disclosed above. Rather, the specific features and acts described herein are disclosed as illustrative implementations of the claims.

1. A system comprising:
at least a portion of a process system being configured to process a fluid, the fluid to have a shear rate at a point of interest in the process system; and
a sensor platform in fluid communication with the process system, the sensor platform further comprising
a port configured to receive the fluid from the process system;
a valve and a sensor set in communication with the port; and
a platform controller in communication with a process controller of the process system, the sensor set, and the valve, the controller being configured to receive a sensed flow rate of the fluid from the process controller and to control the valve so as to duplicate the shear rate of the fluid at the point of interest in the process system, the platform controller being further configured to sense a property of the fluid at the duplicated shear rate via the sensor set and to output the property of the fluid at the sensor set and at the duplicated shear rate where by the platform duplicates conditions in the process system at the point of interest and whereby the sensor set senses the fluid property under conditions substantially similar to those at the point of interest in the process system.

2. A system comprising:
a port configured to receive a fluid to have a shear rate in a process system;
a valve and a sensor set in communication with the port; and
a controller in communication with the process system, the sensor set, and the valve, the controller being configured to receive a sensed flow rate of the fluid from the process system and to control the valve so as to duplicate the shear rate of the fluid in the process system, the controller being further configured to sense a property of the fluid at the duplicated shear rate via the sensor set and to output the property of the fluid at the sensor set and at the duplicated shear rate.

3. The system of claim 2 further comprising first and second spools, the sensor set being on the first spool, the system further comprising a pH and ORP sensor on the second spool and in communication with the controller whereby the controller can sense the pH and ORP of the fluid and close the valve if the pH is outside of a user selected range.

4. The system of claim 2 further comprising first and second spools, the sensor set being on the first spool, the system further comprising an oxygen reduction potential sensor on the second spool and in communication with the controller whereby the controller can sense the oxygen reduction potential of the fluid and close the valve if the oxygen reduction potential is outside of a user selected range.

5. The system of claim 2 further comprising a feedback loop in communication with the controller and the process system, the controller being further configured to send a control signal to the process system which is indicative of a control action to take based on the value of the sensed property of the fluid.

6. The system of claim 2 wherein the port, the valve, the sensor set, and the sensed property are a first port, a first valve, a first sensor set, and a first sensed property, the system further comprising a second port, a second valve, and a second sensor set, wherein the first port is in communication with the process system upstream of a blender and the second port is in communication with the process system downstream of the blender and where in the controller if further configured to sense a second fluid property via the second sensor set and to output an indication thereof.

7. The system of claim 6 wherein the first and second sensed properties are of a same type.

8. The system of claim 7 wherein the controller is further configured to determine and to output a signal indicative of the difference between the first and second sensed properties.

9. The system of claim 6 further comprising a solids separator upstream of the second sensor set.

10. The system of claim 2 wherein the sensor set is on a spool with a vertical orientation.

11. The system of claim 2 wherein the sensor set further comprises one or more sensors selected from a group consisting of a viscosity sensor, a corrosion sensor, a conductivity sensor, a flow meter, a turbidity sensor, or a fluid density sensor.

12. The system of claim 2 wherein the fluid is fracking water.

13. A method of monitoring a fluid in a process system associated with a well, the process system having a main line for providing the fluid to the well, comprising the steps of:
Determining a shear rate of the fluid flowing in the process system line;
Diverting some of the fluid from the main line into a secondary line;
Flowing the fluid through the secondary line at the same shear rate as the fluid flowing in the process system main line;
Measuring a property of the diverted fluid flowing in the secondary line;
Returning the diverted fluid to the process system main line.

14. The method of claim 13 wherein the step of diverting some of the fluid from the main line into a secondary line further comprises the step of diverting some of the fluid from the main line at a location downstream of a blender into a first secondary line and the step of returning the diverted fluid to the process system main line further comprises the step of returning the diverted fluid to the process system main line downstream of the blender.

15. The method of claim 14 further comprising the steps of:
Determining a shear rate of the fluid in the process system main line at a location upstream of the blender;
Diverting some of the fluid from the main line at the location upstream of the blender into a second secondary line;
Flowing the fluid through the second secondary line at the same shear rate as the fluid flowing in the process system main line at the second location;
Measuring the property of the fluid flowing in the second secondary line;
Returning the diverted fluid to the process system main line at a location upstream of the blender.

16. The method of claim 13 wherein the fluid comprises water, further comprising the step of adding a proppant to the water with the blender.

17. The method of claim 13 wherein the step of flowing the fluid through the secondary line at the same shear rate of the fluid in the process system line further comprises controlling a valve in the secondary line.

18. The method of claim 13 further comprising the steps of:
Diverting some of the fluid from the main line into a holding spool;
Capturing the fluid in the holding spool for a predetermined period of time;
Measuring a second property of the fluid in the holding spool after the period of time;
Returning the captured fluid to the process system main line.

19. The method of claim 18 wherein the step of measuring a second property of the fluid in the holding spool further comprises the step of measuring at least one of the properties selected from a group consisting of pH or oxygen reduction potential.

20. The method of 13 further comprising sending a signal from a platform controller to the process system indicative of a control action to take based on the value of the sensed property of the fluid.

21. The method of claim 13 wherein the step of measuring a property of the fluid flowing in the secondary line further comprises the step of measuring at least one of the properties selected from a group consisting of corrosion, conductivity, pH, temperature or pressure.
